(19) United States
(12) Patent Application Publication
(10) Pub. No.: US 2026/0051073 A1
(43) Pub. Date: Feb. 19, 2026

(54) Plant Phenotyping

(71) Applicant: C-Crop LTD., Mazkeret Batia (IL)

(72) Inventors: Ilya Leizerson, Haifa (IL); Hagai Karchi, Sitria (IL); Netanel Ben Iluz, Nahariya (IL); Raanan Ganor, Bnei-Atarot (IL); Zur Granevitze, Mazkeret Batia (IL)

(21) Appl. No.: 19/115,885

(22) PCT Filed: Sep. 27, 2023
(86) PCT No.: PCT/IL2023/051041

§ 371 Date: Mar. 27, 2025

(30) Foreign Application Priority Data
Sep. 29, 2022 (IL) 296946

(51) Int. Cl. G06T 7/55; G01N 21/84; G01S 17/89; G06T 7/00; G06T 7/70

(52) U.S. Cl. CPC G06T 7/55; G01N 21/84; G01S 17/89; G06T 7/0014; G06T 7/70; G01N 2021/8466; G06T 2207/10028; G06T 2207/30188; G06T 2207/30242

(57) ABSTRACT

Method and system for plant phenotyping. Sequence of images of agricultural scene are captured along trajectory using image sensor of mobile computing device. Position and orientation data is obtained for each captured image using inertial sensor of mobile computing device. Point cloud generated in captured images, point cloud including coordinates defining distances to respective features in image. An object is selected, object belonging to plant hierarchy level of: plant organ; plant; plant grouping; or plant field. For each selected first object in first plant hierarchy level, object is identified and tracked in successive images of sequence, distance coordinates for object are supplemented when point cloud insufficient; visually undetectable portions of object are supplemented in images; spatial distance of object in respective images is determined based on point cloud coordinates and supplemented distance coordinates; and at least one phenotype of object is determined, based on determined spatial distance of object.

### TECHNICAL FIELD

The present disclosure relates to agricultural productivity and crop analysis in general, and to data processing and evaluation for determining phenotypes and phenotype statistics of plant crops in particular.

### BACKGROUND

Evaluation of crops is integral for improving yields and enhancing productivity in agricultural, horticultural and aquaculture environments. Information gleaned about the crops can be used to influence decisions relating to cultivation, growth and harvesting of a multitude of crop variants. A crop yield is the harvested production per unit of harvested area. The actual yield generally depends on several factors, such as genetic characteristics of the crop, amount and duration of sunlight, water and nutrients absorbed by the crop, and influence of pests or diseases. Crop parameters and characteristics, such as the size or amount of individual fruits in a batch or in a crop segment or a field section, can be used for determining optimal growth or harvesting actions, such as whether to dilute the amount of crop plants in a selected region.

A “phenotype” refers to a set of observable characteristics or traits of an organism, which may be a human, an animal or a plant. A plant phenotype can include basic traits, such as the size, shape, or color of individual crop fruits, or supplementary traits, such as the presence or absence of diseases or other irregularities. Important crop parameters include those relating to an individual crop plant, such as dimensions, shape or color of an individual fruit or leaf, as well as parameters pertaining to a group of plants, such as the amount, the density, or the uniformity of fruits in a given plant cluster. It is further noted that some phenotypes may be visible, while others may be hidden or imperceptible to an ordinary observer.

The use of imaging and image analysis to facilitate crop evaluation is well known. Images of crop fields captured by sensors can be analyzed using automated processing tools to derive useful information pertaining to the crops. However, in some circumstances there may be a significant discrepancy between the information extracted from imaging devices and the real conditions that actually exist in the field. Important information may be missed or neglected in obtained images due to a limited viewing angle or sub-optimal conditions at which the image was captured. For example, a crop portion of interest may be obscured or obstructed by a different crop segment in the image foreground. The position at which the image sensor is situated may preclude imaging of the entire agricultural field such that some areas may be imaged ineffectually or not at all. Extraction of certain crop parameters may also be impeded by environmental or climate factors, such as intense precipitation, fog or sunlight. Moreover, it may be difficult to differentiate between fruits or clusters of different types in an image, as the nuances may be exceedingly subtle.

Furthermore, the imaging data may only reveal part of a plant phenotype or only selected phenotypes, while overlooking other phenotypes or phenotype portions. The high density of plants and plant organs in common crop species may lead to phenotyping errors, such as due to merged or split plant organs, or overlapping portions from different plants creating a confusing observation. It is also necessary to distinguish between plant organs that are relevant to a considered phenotype and those that are not relevant, for example to distinguish leaves and inflorescences from fruits, or to distinguish between fruits from different plants located in different plantation rows (e.g., to prevent overcounting).

Publications describing the measurement and analysis of plant phenotypes include the following:

Liu, L., Yu, L., Wu, D., Ye, J., Feng, H., Liu, Q., & Yang, W. (2021). PocketMaize: An Android-Smartphone Application for Maize Plant Phenotyping. Frontiers in Plant Science, 12. discloses a portable whole-plant on-device phenotyping smartphone application running on Android that can measure up to 45 traits, including 15 plant traits, 25 leaf traits and 5 stem traits, based on images. A DeepLabV3+ model for segmentation was trained to avoid the influence of outdoor environments, and an angle calibration algorithm was designed to reduce error introduced by different imaging angles.

U.S. Patent Application Publication No. 2020/0294620 to Bauer et al, entitled: “Method and system for performing data analysis for plant phenotyping”, is directed to a method and a data acquisition system for performing data analysis for single plants in a field, and mobile platform therefor. The method comprises the steps of comprises the steps of capturing spectral data via a hyperspectral imaging sensor, capturing image data via an image sensor, capturing georeference data via an inertial measurement unit, spatializing the image data to generate georeferenced image data and a digital surface model, spatializing the spectral data, generating georeferenced spectral data based on the spatialized spectral data and the digital surface model and overlaying the georeferenced image data and georeferenced spectral data with field plan information to generate a high-resolution analysis data set.

China Patent No. CN112200854 (A) to South China Agricultural University, entitled: “Leaf vegetable three-dimensional phenotype measurement method based on video image”, discloses a leaf vegetable three-dimensional phenotype measurement method based on a video image. The method comprises the following steps: acquiring video image data of a leaf vegetable through a data acquisition device; performing blurred image frame removal processing on the video image data, and obtaining a key frame containing a leaf vegetable region in the video image data by using a transformation matching method based on a vegetation index and a scale invariant feature; reconstructing the key frame image into a three-dimensional point cloud model, and performing post-processing of a three-dimensional space through the three-dimensional point cloud model to obtain a post-processing point cloud model; and extracting a point cloud skeleton from the post-processing point cloud model, conducting point cloud segmentation, then calculating leaf vegetable phenotype parameters.

U.S. Pat. No. 9,886,749 to Schmitt et al, entitled: “Apparatus and method for parameterizing a plant”, is directed to the parameterization of plants for agricultural technology. The method includes the steps of: recording a three-dimensional data set of the plant, which does not only include volume elements of non-covered elements of the plant, but also volume elements of elements of the plant that are covered by other elements; and parameterizing the three-dimensional data set for acquiring plant parameters, where parameterizing includes: converting the three-dimensional data set into a point cloud, where the point cloud only includes points on a surface of the plant or points of a volume structure of the plant, segmenting the three-dimensional point cloud into single elements of the plant, where a single element is a leaf, a stem, a branch, a trunk, a blossom, a fruit or a leaf skeleton, and calculating, by using a single-element model, parameters for the single element by adapting the single-element model to the single element.

Japan Application Publication No. JP 2022089140A to UNIV ZHEJIANG, entitled: “Field plant phenotypic information collection system and method”, discloses a phenotypic information collection system arranged on a self-propelled field carrier. The field plant phenotypic information collection system comprises a controller, and a sensor group, a GPS module and a wireless communication module which are connected with the controller. The sensor group is used for collecting phenotypic information of field crops; wherein the phenotypic information comprises RGB image information, plant-form three-dimensional point cloud data and hyperspectral data. The GPS module is used for acquiring real-time geographic information of the self-propelled field carrier. The controller is used for controlling opening and closing of the sensor group according to information collection position data and real-time geographic information input by a user of a ground control center, generating a preview from the phenotypic information and sending the preview to the ground control center through the wireless communication module.

An overview of 3D plant phenotyping methods posted on 8 Oct. 2019 in phenotyping methods by Stefan Schwartz can be found at: https://phenospex.com/blog/an-overview-of-3d-plant-phenotyping-methods/.

### SUMMARY

In accordance with one aspect of the present disclosure, there is thus provided a method for plant phenotyping. The method includes the procedure of capturing a sequence of images of an agricultural scene from a plurality of positions and orientations along a trajectory using at least one image sensor of a mobile computing device, and obtaining position and orientation data for each captured image using at least one inertial sensor of the mobile computing device. The method further includes the procedures of generating a point cloud in each of the captured images, the point cloud comprising at least one coordinate defining a distance from the image sensor to a respective feature in the image, and selecting at least one object belonging to a plant hierarchy level comprising one of: a plant organ; a plant; a grouping of plants; and a field of plants. The method further includes, for each of at least one first selected object in a first plant hierarchy level, applying the processing steps of: identifying and tracking the object in successive images of the sequence; supplementing distance coordinates for the object when the point cloud is insufficient; supplementing visually undetectable portions of the object in the images; determining a spatial distance of the object in respective images based on the point cloud coordinates and the supplemented distance coordinates; and determining at least one phenotype of the object, based on the determined spatial distance of the object. The method may further include procedures of associating at least one determined phenotype with a reliability metric reflecting a degree of confidence or reliability of the determined phenotype, and determining at least one phenotype statistic respective of the reliability metric. The method may further include the procedure of grouping a plurality of selected objects into at least one category, and determining at least one phenotype or phenotype statistic relating to the category. The method may include selecting at least one second selected object in a second plant hierarchy level, and applying the processing steps for the second selected object. The method may further include the procedure of providing a phenotyping report on a phenotype application executing on the mobile computing device, the phenotyping report comprising information relating to at least one of: at least one determined phenotype; at least one reliability metric; at least one category; and at least one phenotype statistic. The method may further include the procedure of obtaining environmental data of the agricultural scene, where at least one of the procedures of: supplementing distance coordinates; supplementing visually undetectable portions of the object; and determining at least one phenotype, is performed based on the environmental data. The procedure of supplementing distance coordinates may be performed using at least one technique of: Light Detection and Ranging (LIDAR); stereoscopic imaging; determining an average or accepted distance for a same or similar object; and/or extrapolating from distance information in at least one other image. The method may further include the procedure of reconstructing an imaging trajectory of the image sensor when capturing the sequence of images, using the position and orientation data or the captured images, and using the reconstructed imaging trajectory for at least one of: determining or updating a distance measurement from the image sensor to the object; correcting a position and orientation of the image sensor; and determining an alignment of an arrangement of plants. The method may further include the procedure of providing at least one recommendation for optimizing at least one selected attributes of crop development in accordance with the determined phenotype information, on a phenotype application executing on the mobile computing device. The phenotype may include at least one attribute of: size; shape; dimensions; volume; amount; density; color; regularity; uniformity; developmental stage; and/or presence or absence of at least one pest or at least one disease or plant disorder. The reliability metric may be determined based on at least one parameter selected from the group consisting of: number or reliability of distance coordinates for object; number of plant organ obstructions; number or degree of obstructions of object in images; size, distance or regularity of object in images; and imaging characteristics of the image sensor. At least one of the processing steps of: supplementing distance coordinates for the object; and supplementing visually undetectable portions of the object, may be applied based on an intactness metric reflecting a degree to which the object is well-defined in a respective image. The sequence of images may be captured while moving the mobile computing device along a trajectory by a process of: at least one person manually conveying the mobile computing device, and/or a moving platform repositioning the mobile computing device. The sequence of images may be captured from a minimum imaging distance, such that the object occupies a minimum number of image pixels in the captured image. The method may further comprise the procedure of providing at least one recommendation for guiding the imaging of an object in accordance with the spatial distance of the object in relation to the imaging sensor of the mobile computing device.

In accordance with another aspect of the present disclosure, there is thus provided a system for plant phenotyping. The system includes a mobile computing device, communicatively coupled to a computer network. The mobile computing device includes at least one image sensor, configured to capture a sequence of images of an agricultural scene from a plurality of positions or orientations along a trajectory relative to the agricultural scene. The mobile computing device includes at least one inertial sensor, configured to obtain position and orientation data along the trajectory, when the mobile computing device is moved along the trajectory. The system further includes a processor, configured to generate a point cloud in each of the captured images, the point cloud comprising at least one coordinate defining a distance from the image sensor to a respective feature in the image, and to select at least one object belonging to a plant hierarchy level comprising one of: a plant organ; a plant; a grouping of plants; and a field of plants. For each selected first object in a first plant hierarchy level, the processor is further configured to: identify and track the object in successive images of the sequence; to supplement distance coordinates for the object when the point cloud is insufficient; to supplement visually undetectable portions of the object in the images; to determine a spatial distance of the object in respective images based on the point cloud coordinates and the supplemented distance coordinates; and to determine at least one phenotype of the object, based on the spatial distance of the object. The processor may be further configured to associate at least one determined phenotype with a reliability metric reflecting a degree of confidence or reliability of the determined phenotype, and to determine at least one phenotype statistic respective of the reliability metric. The processor may be further configured to group a plurality of objects into at least one category, and to determine at least one phenotype or phenotype statistic relating to the category. The system may include selecting at least one second selected object in a second plant hierarchy level, and applying the processing steps for the second selected object. The system may further include a phenotype application executing on the mobile computing device, the phenotype application configured to provide a phenotyping report comprising information relating to at least one of: at least one determined phenotype; at least one reliability metric; at least one category; and at least one phenotype statistic. The system may further include at least one environmental sensor, configured to obtain environmental data of the agricultural scene, where at least one of: supplementing distance coordinates; supplementing visually undetectable portions of the object; and determining at least one phenotype, is performed based on the environmental data. The processor may be configured to supplement distance coordinates using at least one technique of: Light Detection and Ranging (LIDAR); stereoscopic imaging; determining an average or accepted distance for a same or similar object; and/or extrapolating from distance information in at least one other image. The processor may be configured to reconstruct an imaging trajectory of the image sensor when capturing the sequence of images, using the position and orientation data or the captured images, and use the reconstructed imaging trajectory for at least one of: determining or updating a distance measurement from the image sensor to the object; correcting a position and orientation of the image sensor; and determining an alignment of an arrangement of plants. The phenotype application may be configured to provide at least one recommendation for optimizing at least one selected attributes of crop development in accordance with the determined phenotype information. The phenotype may include at least one attribute of: size; shape; dimensions; volume; amount; density; color; regularity; uniformity; developmental stage; and/or presence or absence of at least one pest or at least one disease or plant disorder. The reliability metric may be determined based on at least one parameter selected from the group consisting of: number or reliability of distance coordinates for object; number of plant organ obstructions; number or degree of obstructions of object in images; size, distance or regularity of object in images; and imaging characteristics of the image sensor. At least one of the processing steps of: supplementing distance coordinates for the object; and supplementing visually undetectable portions of the object, may be applied based on an intactness metric reflecting a degree to which the object is well-defined in a respective image. The sequence of images may be captured from a minimum imaging distance, such that the object occupies a minimum number of image pixels in the captured image. The phenotype application may further be configured to provide at least one recommendation for guiding the imaging of an object in accordance with the spatial distance of the object in relation to the imaging sensor of the mobile computing device.

### BRIEF DESCRIPTION OF THE DRAWINGS

The present disclosure will be understood and appreciated more fully from the following detailed description taken in conjunction with the drawings in which:

FIG. 1 is a schematic illustration of a network environment supporting a system for plant phenotyping, constructed and operative in accordance with an embodiment of the present disclosure;

FIG. 2 is an illustration of imaging a plant field for plant phenotyping, operative in accordance with an embodiment of the present disclosure;

FIG. 3 is a schematic illustration of a camera capturing a sequence of images at different positions and orientations along a trajectory, operative in accordance with an embodiment of the present disclosure;

FIG. 4A is a first exemplary image containing a point cloud and captured for plant phenotyping, in accordance with an embodiment of the present disclosure;

FIG. 4B is a second exemplary image containing a point cloud and captured for plant phenotyping, in accordance with an embodiment of the present disclosure;

FIG. 5A is a first graph showing an exemplary phenotype measurement for grape cluster length obtained by a method for plant phenotyping operative in accordance with an embodiment of the present disclosure, as a function of the same phenotype measurement obtained manually, for a first reliability metric;

FIG. 5B is a second graph showing an exemplary phenotype measurement for grape cluster length obtained by a method for plant phenotyping operative in accordance with an embodiment of the present disclosure, as a function of the same phenotype measurement obtained manually, for a second reliability metric; and

FIG. 6 is a flow diagram of a method for plant phenotyping, operative in accordance with an embodiment of the present disclosure.

### DETAILED DESCRIPTION OF THE EMBODIMENTS

The present disclosure overcomes the disadvantages of the prior art by providing methods and systems for plant phenotyping, that provides accurate and comprehensive phenotype measurements of plants reflective of their actual state, based on multi-image data, allowing for augmentation of crop development processes and improving crop yield and productivity. Plant phenotype measurements may be obtained for different hierarchical levels, including for an individual plant, for a portion of the plant (such as a plant organ), for a group of plants (such as a plant fruit cluster), and for an entire field.

Unless otherwise defined, all terms (including technical and scientific terms) used herein have the same meaning as commonly understood by one of ordinary skill in the art to which the present disclosure pertains. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

It will be understood that when an element is referred to as being “on”, “attached” to, “operatively coupled” to, “operatively linked” to, “operatively engaged” with, “connected” to, “coupled” with, “contacting”, “added to, another element, it can be directly on, attached to, connected to, operatively coupled to, operatively engaged with, coupled with, added to, and/or contacting the other element or intervening elements can also be present. In contrast, when an element is referred to as being “directly contacting” another element or “directly added” to another element, there are no intervening elements and/or steps present.

Whenever the term “about” or “approximately” is used, it is meant to refer to a measurable value such as an amount, a temporal duration, and the like, and is meant to encompass variations (e.g., ±20%, ±10%, ±5%, ±1%, ±0.1%) from the specified value, as such variations are appropriate to perform the disclosed embodiments.

Certain features of the present disclosure, which are, for clarity, described in the context of separate embodiments, may also be provided in combination in a single embodiment. Conversely, various features of the present disclosure, which are, for brevity, described in the context of a single embodiment, may also be provided separately or in any suitable sub-combination or as suitable in any other described embodiment of the present disclosure. Certain features described in the context of various embodiments are not to be considered essential features of those embodiments, unless the embodiment is inoperative without those elements.

Throughout this application, various embodiments of the present disclosure may be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range, regardless of the breadth of the range. Whenever a numerical range is indicated herein, it is meant to include any cited numeral (fractional or integral) within the indicated range. For example, the phrases “ranging/ranges between” a first indicated number and a second indicated number and “ranging/ranges from” a first indicated number “to” a second indicated number are used herein interchangeably and are meant to include the first and second indicated numbers and all fractional and integral numerals there between.

Whenever terms “plurality” and “a plurality” are used it is meant to include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

The terms “crop” and “plant” are used interchangeably herein to refer to a multicellular living organism capable of performing photosynthesis (i.e., excluding humans and animals) and which undergoes growth and harvesting processes, which may (but not necessarily) bear edible fruits (i.e., “food crops and feed crops”), leaves or other plant organs, and also encompasses energy crops (i.e., crops grown for energy production, such as oil producing crops). Examples of crops may include, but are not limited to: cereal crops, such as rice, corn, wheat and barley; seed crops, such as grains, legumes and nuts; fruit crops, such as: apples, bananas, grapes, oranges, and peaches; and vegetable crops, such as potatoes, carrots, and lettuce. A “plant organ” refers to an individual segment, or portion thereof, of a single plant, during any developmental stage, including but not limited to: a root, a stem, a leaf, a flower, a seed, or a fruit.

The term “field” as used herein refers to an area of land or property in which crops are grown and cultivated, such as: a farming or gardening plot; a greenhouse or other enclosed structure for regulating plant growth environment, an enclosed building for vertical farming; and may also include bodies of water, such as for aquatic plant growth.

The term “phenotype” is used herein to broadly refer to any characteristic, parameter or trait of a plant or crop, including those relating to an individual plant, to segments of a plant (i.e., plant organ), or to a group of plants (e.g., in an entire field), and including visible and perceptible traits as well as hidden or imperceptible traits. Examples of phenotypes may include, but are not limited to: size (dimensions), shape, or color of a plant, a plant organ, or a plant grouping; the number or density or uniformity of fruits in a single plant or in a plant grouping in different development stages and health conditions (e.g., phenotype symptoms in response to biotic and abiotic stresses such as water deficiency or nutrient deficiency; the presence of insects, pests or plant-related diseases; and the like. It is noted that a phenotype measurement is dependent on whether the trait relates to an individual plant, a plant organ, or a group of plants, and a given phenotype may vary significantly between similar plants in the same field.

The terms “user” and “operator” are used interchangeably herein to refer to any individual person or group of persons using or operating a method or system for plant phenotyping in accordance with the present disclosure.

The term “mobile platform”, and any variations thereof, as used herein refers to any platform or surface capable of moving from one location to another, including, but not limited to: a vehicle, a transportation medium, or a person. Accordingly, a mobile computing device of the present disclosure may be mounted on and transported by a vehicle or other movable platform, or may be directly conveyed by at least one person.

The term “repeatedly” as used herein should be broadly construed to include any one or more of: “continuously”, “periodic repetition” and “non-periodic repetition”, where periodic repetition is characterized by constant length intervals between repetitions and non-periodic repetition is characterized by variable length intervals between repetitions.

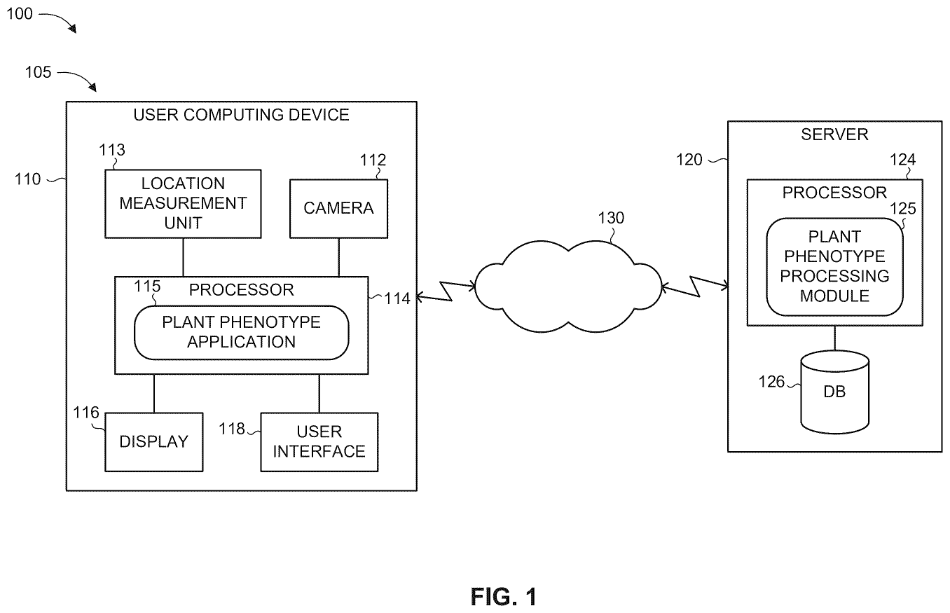

Reference is now made to FIG. 1, which is a schematic illustration of a network environment, generally referenced 100, supporting a computer-implemented system, generally referenced 105, for plant phenotyping, constructed and operative in accordance with an embodiment of the present disclosure. Environment 100 includes at least one user computing device 110, and, optionally, at least one server 120. User computing device 110 includes at least one camera 112, an inertial measurement unit (IMU) 113, a processor 114, a display 116, and a user interface 118. Server 120 includes a processor 124 and a database 126. System 105 includes a plant phenotyping application 115 operating on user computing device processor 114, and a plant phenotyping processing module 125 operating on server processor 124, although it is appreciated that the functionality of any of the system modules may operate on either or both of user computing device 110 or server 120.

User computing device 110 and server 120 are communicatively coupled through at least one network 130, such as the Internet. Accordingly, information may be conveyed between user computing device 110 and server 120, as well as to/from other networks communicatively coupled thereto, over any suitable data communication channel or network, using any type of channel or network model and any data transmission protocol (e.g., wired, wireless, radio, WiFi, Bluetooth, and the like). For example, images and other collected data may be uploaded and dynamically processed in real-time using a cloud computing platform. User computing device 110 may be remotely located from server 120. Network environment 100 may include a plurality of user computing devices operated by multiple respective operators, although a single user device 110 is depicted for exemplary purposes. Similarly, network environment 100 may include a plurality of remote servers, or alternatively may operate without a server using only a user computing device, but a single server 120 is depicted for exemplary purposes.

User computing device 110 may be embodied by any type of electronic device with computing and network communication capabilities, including but not limited to: a smartphone; a laptop computer; a mobile computer; a netbook computer; a tablet computer; or any combination of the above. User computing device 110 is mobile or portable, and configured to be conveyed to different locations, such as via at least one person (i.e., a user), via a transportation medium and/or via a mobile platform. For example, user computing device 110 may be transported using a vehicle (i.e., constituting a mobile computing device) or may be held and repositioned by a user (i.e., constituting a portable computing device).

User computing device 110 includes, or is coupled with, a camera 112. Camera 112 may be any type of imaging sensor capable of acquiring and storing an image representation of a scene, such as of an agricultural field. Accordingly, the term “image” as used herein refers to any form of output from an aforementioned camera, including any optical or digital representation of a scene acquired at any wavelength or spectral region (e.g., visible or infrared), and encompasses both a single image frame and a sequence of image frames (i.e., a “video image”). Computing device 110 may include multiple cameras, such as a visible light (e.g., RGB) imaging sensor and an infrared (e.g., IR, NIR, SWIR, LWIR) imaging sensor).

User computing device 110 includes, or is coupled with, an inertial measurement unit (LMU) 113 that provides an indication of the location (i.e., position and orientation) of computing device 110 or associated components, such as a viewing direction of camera 112 and/or position and orientation coordinates of a scene imaged by camera 112. IMU 113 may be embodied by one or more sensors or instruments configured to measure the position and orientation of computing device 112 with respect to a reference coordinate system, including but not limit to: a global positioning system (GPS); an inertial navigation system (INS); motion sensors or rotational sensors (e.g., accelerometers, gyroscopes, magnetometers); a compass; a rangefinder; a camera; and the like. IMU 113 may include one or more pre-existing sensors or instruments of mobile computing device 110, such as inertial sensors or GPS sensors inherent in standard smartphones. IMU 113 may provide position and orientation measurements with respect to any reference coordinate system defined in relation to user computing device 110.

User computing device 110 further includes, or is coupled with, a display 116 that is configured to present visual content, such as a display screen. User computing device 110 further includes, or is coupled with, a user interface 118 that allows the user to control parameters or settings associated with computing device 110. User interface 118 may include a cursor and/or a touch-screen menu interface, such as a graphical user interface, configured to enable manually entering instructions or data. User interface 118 may also include peripheral communication devices configured to provide voice communication, such as a microphone and an audio speaker, as well as voice recognition capabilities to enable the user to enter instructions or data by means of speech commands.

Processor 114 performs data processing required by user computing device 120, and may receive instructions or data from other components of system 105 or network environment 100. For example, plant phenotyping application 115 operating on processor 114 analyzes and processes image data obtained from camera 112 and inertial data obtained from LMU 113, as will be discussed further hereinbelow. Server processor 124 performs necessary data processing required by server 120, and may receive instructions or information from other components of system 105 or network environment 100, such as from user computing device 120.

Database 126 stores relevant information that can be retrieved and managed by plant phenotyping application 115, such as image data, location data, and determined phenotype parameters and classifications. Alternatively or additionally, information may be stored in a local memory (not shown) of user computing device 110.

The components and devices of system 105 may be based in hardware, software, or combinations thereof. It is appreciated that the functionality associated with each of the devices or components of network environment 100 or system 105 may be distributed among multiple devices or components, which may reside at a single location or at multiple locations. For example, the functionality associated with processor 114 or processor 124 may be integrated or may be distributed between multiple processing units. Similarly, at least part of the functionality associated with plant phenotyping application 115 may reside externally to user computing device 110 or server 120. System 105 may optionally include and/or be associated with additional components or modules not shown in FIG. 1, for enabling the implementation of the disclosed subject matter.

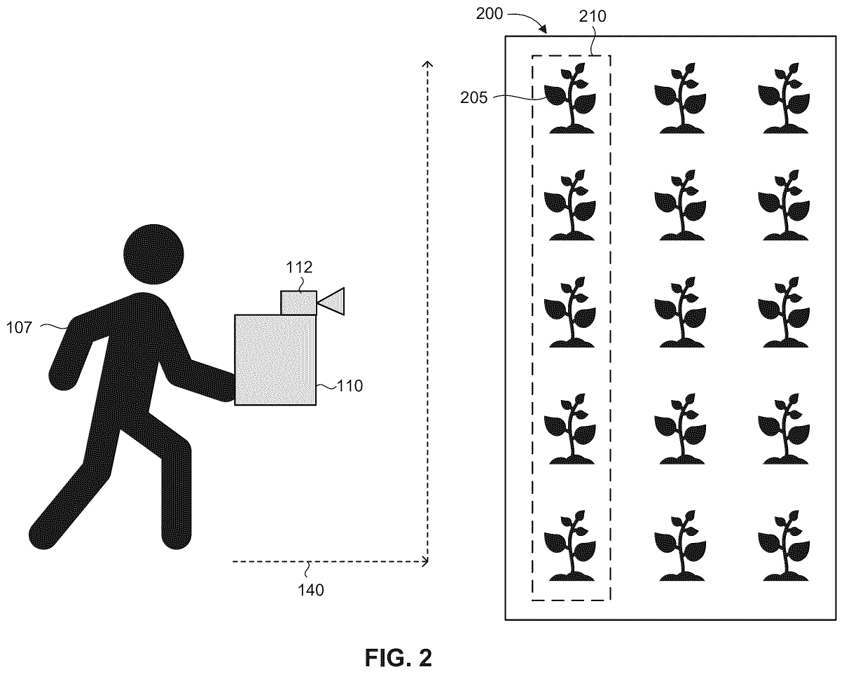

The operation of system 105 will now be described in general terms, followed by specific examples. A user is located near an agricultural scene of interest for which plant phenotypes are to be determined, such as a field containing one or more selected crop plants (e.g., an apple orchard). The term “agricultural scene” is used herein to refer to a section of a field selected for plant phenotyping. Application 115 may be provided with relevant information relating to the selected field prior to the phenotyping, such as a name or identifier of the field; type of field; size of field; geographic location; date and start time of phenotyping; and the like. Application 115 may obtain field details directly from the user (via user interface 118) or may retrieve details from computing device 110 or publicly available online sources. Reference is made to FIG. 2, which is an illustration of imaging a plant field for plant phenotyping, operative in accordance with an embodiment of the present disclosure. A user, referenced 107, is holding a computing device 110 with a camera 112 and following a path 140 alongside a plant field 200. Field 200 includes a plurality of plants, referenced 205, which are arranged in various plant groupings 210. Each plant 205 is made up of individual plant organs (e.g., leaves, stems, roots, flowers, fruits).

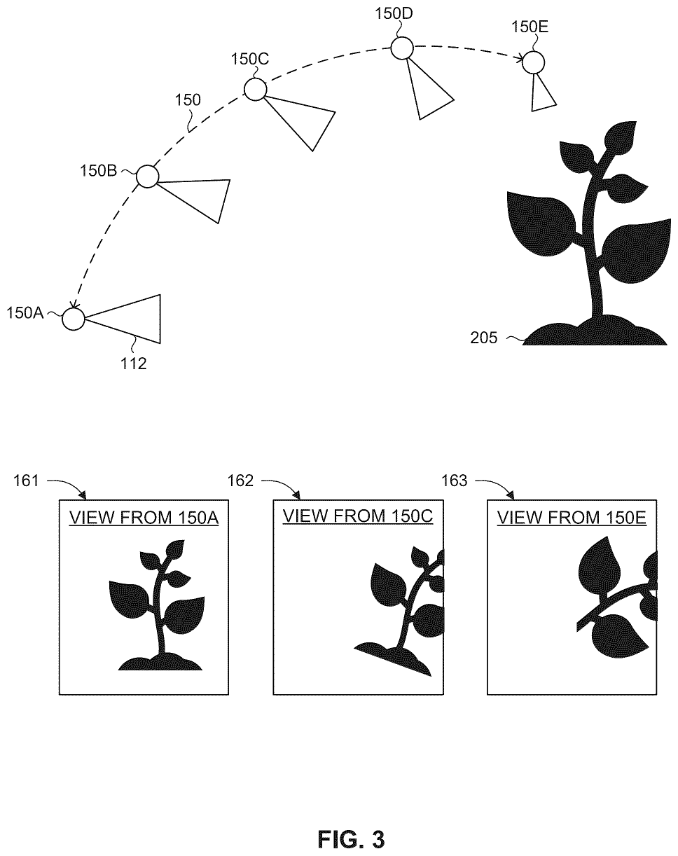

To initiate the phenotyping method, camera 112 is directed to obtain a sequence of images of the agricultural scene at a plurality of positions and orientations (viewing angles). In particular, camera 112 is conveyed along a selected trajectory relative to the agricultural scene while capturing a series of image frames at different points along the trajectory. For example, camera 112 may be held by a user 107 and repositioned along the trajectory points, or many be mounted on a mobile platform, such as a vehicle or a robotic trolley, and transported to different positions along the trajectory. Reference is made to FIG. 3, which is a schematic illustration of a camera capturing a sequence of images at different positions and orientations along a trajectory, operative in accordance with an embodiment of the present disclosure. Camera 112 is moved along trajectory 150 and acquires images of a plant crop 205 at each of trajectory points 150A, 150B, 150C, 150D and 150E, at respective positions and orientations in relation to the agricultural scene. For example, captured image 161 depicts an imaging viewpoint from trajectory point 150A; captured image 162 depicts an imaging viewpoint from trajectory point 150C; and captured image 163 depicts an imaging viewpoint from trajectory point 150E. It is noted that five trajectory points are depicted for exemplary purposes only, although a large number of images should be obtained along the trajectory, at an image frame rate sufficient to ensure adequate coverage. For example, camera 112 may acquire at least 90 images of a selected scene and operate at a frame rate of 30 Hz/fps along a trajectory distance of 5 m. Trajectory 150 may follow a straight path (e.g., parallel or perpendicular to the scene) or an angular/non-orthogonal path relative to the scene, and may consist of one or more sub-trajectories, such as by repositioning the camera multiple times back and forth along a designated path. In general, the movement pattern followed by camera 112 may be predetermined or may be spontaneously established by the user or mobile platform, so long as a sufficient number of image frames are obtained at sufficiently varying positions and orientations. The imaging trajectory is established such that camera 112 is positioned no further than a minimum distance relative to features of interest (e.g., plants and plant organs) in the scene. The minimum imaging distance is generally a function of the crop fruit size, such that a smaller crop fruit requires imaging from a shorter distance. For example, the minimum imaging distance may be approximately 1 to 3 meters when imaging a grapevine crop, and slightly further (e.g., 3 to 6 meters) when imaging an apple crop. More generally, the minimum imaging distance relative to a plant or plant organ may be established as a function of the plant or plant organ size, such that the plant or plant organ should occupy a minimum number of image pixels (e.g., at least 10 image pixels) in the captured image.

Each captured image is associated with inertial or location data obtained from IMU 113 of computing device 110. In particular, IMU 113 provides an indication of the position and orientation of computing device 110 corresponding to a position and viewing direction of camera 112 while capturing the respective image. Each captured image is further associated with metadata, which may include imaging characteristics of the camera, such as: focal length, lens type, field of view, resolution, sensitivity, pixel size, dynamic range, and/or operating frequencies, as well as general parameters relating to the respective image, such as: date, time and/or geographic location of imaging.

The images and associated location (position and orientation) data and metadata are stored and processed. In particular, application 115 processes a sequence of images to identify an object of interest, such as a plant organ of the imaged plant. The images may be processed substantially in real-time (e.g., immediately following their imaging) or may include previously captured images, such as days, weeks, or months previously (e.g., which are stored and uploaded in server database 126). The images may optionally undergo pre-processing, such as if images are obtained from multiple cameras and/or at substantially different times or dates, to provide a uniform format for subsequent analysis or correction of optical aberrations, using image processing techniques known in the art.

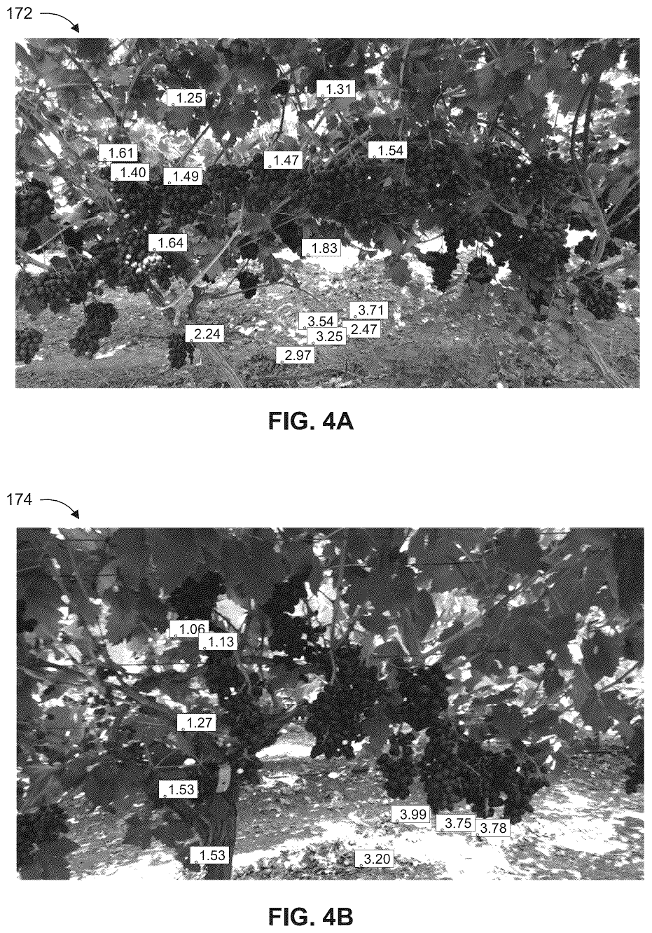

Each image may include a point cloud, defining one or more three-dimensional (3D) spatial coordinates representing at least the distance from features in the imaged scene to the lens of camera 112. The point cloud may be generated by a process operating simultaneously to the image and inertial data capturing, or by serial processing of image frames. The images may undergo pre-processing to generate 3D point cloud data using techniques known in the art, such as visual odometry (VO) or visual inertial odometry (VIO), including feature-based and/or pixel intensity-based VO methods. The number of point cloud coordinates may vary among image frames, such that one image frame may be associated with relative few point cloud coordinates compared to another image frame. Similarly, the number of point cloud coordinates may vary within an individual image frame, such that certain image features or portions of an image may be associated with relatively few point cloud coordinates in relation to other image features or portions of the same image. The point cloud may consist of only a single coordinate value reflecting a distance to a feature or object in the scene. The point coordinates may be dense enough to partially or completely delineate the shape of objects. The actual formation of point clouds and their coverages of objects is dependent upon various factors, such as field of view (FOV), angular resolution, and line of sight between camera 112 and the object. The amount and location of point cloud coordinates (particularly the “z-coordinate” reflecting distance) in a given image, in relation to plant organs and plants in the image, may influence a confidence level or reliability metric of at least some determined phenotypes, as will be discussed further hereinbelow. Reference is made to FIGS. 4A and 4B, which respectively show a first exemplary image 172 (FIG. 4A) and a second exemplary image 174 (FIG. 4B), containing a point cloud and captured for plant phenotyping, in accordance with embodiments of the present disclosure. First image 172 depicts a first (e.g., wide angle) view of an agricultural scene with grape crops, and includes multiple numerical values dispersed throughout the imaged contents (i.e., a point cloud). Each numerical value reflects a distance value (z-coordinate) relative to a respective feature in the scene, such as for example, a value “1.61” reflecting a distance to a grape cluster hanging from a branch that appears at an upper left corner of image 172. Second image 174 depicts a second view of an agricultural scene with grape crops (e.g., which may be part of a common image sequence as image 172), but with fewer point cloud coordinates compared to first image 172 (e.g., 5 point cloud coordinates in total compared to 14 in image 172).

The point cloud coordinates that fall on an object of interest (OOI), such as a plant organ, in the processed image, provide an indication of the 3D spatial position of the OOI in relation to camera 112. However, there may be insufficient point cloud coordinates falling on the OOI region in a given image to allow for a determination of the OOI position (particularly distance), such as if most of the point cloud coordinates are associated with other image regions beyond the OOI. For example, there may be too few point cloud coordinates associated with the object (e.g., if the number of coordinates is below a certain threshold), or the point cloud coordinates may be of poor quality or deficient. In such cases, one or more spatial position coordinates for the OOI may be supplemented, so as to facilitate subsequent phenotype determination. Supplementing position coordinates of the OOI, particularly distance values, may be applied using various tools or techniques known in the art. For example, a distance measurement to the object may be determined using a LIDAR detector (i.e., transmitting a laser to the object and measuring return time of reflected pulse), or a stereoscopic rangefinder (e.g., utilizing principles of binocular vision and stereoscopic imaging to measure distance). Other approaches include deriving a distance measurement based on average or accepted distances for the same or similar objects (e.g., for the same type of plant organs); or extrapolating a distance from other image frames based on the distance to the same or similar objects in those image frames (i.e., for which sufficient point cloud coordinates are present) with suitable modifications. The (at least one) supplemented spatial coordinate is associated with the relevant OOI for the particular image frame.

According to an embodiment the present disclosure, a supplemented distance coordinate of an object may be updated in accordance with the location of an imaging trajectory of camera 112 when capturing the images, where the imaging trajectory may be reconstructed using inertial or location data obtained from LMU 113 (e.g., GPS, INS, IMU, motion or rotational sensors such as accelerometers and gyroscopes) and/or using the captured images. The reconstructed imaging trajectory may also be utilized to modify or correct the position and orientation of camera 112. The reconstructed imaging trajectory may further be used to determine an alignment of a row or arrangement of plants in a field plot.

Referring back to FIGS. 1 and 2, application 115 processes a first image frame to identify a first feature or object of interest (OOI) relating to a plant organ of the imaged plant, such as a leaf or fruit of plant 205. The terms “object” and “object of interest” are used interchangeably herein. After an OOI is detected in a first image frame in an image sequence, the OOI is identified and tracked over subsequent image frames. For example, application 115 identifies a leaf of an apple plant crop in a first image and then identifies and tracks the same leaf over subsequent images. The object tracking may be performed using feature detection and/or other image processing techniques known in the art. Each detected object may be assigned an identifier (ID). The object position in the next image frame may be estimated using a Kalman filter considering the position in previous image frames. The estimated position can then be matched with one of the new predictions, so as to track the object over a sequence of frames. For each image, a distance to the leaf is determined based on the point cloud coordinates, specifically according to the distance value (z-coordinate) closest to the object. If there are insufficient point cloud coordinates falling on the leaf in that image such that the distance to the leaf cannot be sufficiently determined, then distance coordinates of the leaf may be supplemented for that image frame, as discussed hereinabove. It is noted that the leaf may appear and reappear over certain frames in the image sequence (e.g., due to variations in the imaging position/orientation that may cause an obstruction or occlusion, or due to interference from environmental features), but may be nevertheless detected and tracked wherever present. It is further noted that the leaf (or other object) distances are determined over the image frames without necessarily generating a three-dimensional (3D) model of the leaf (object).

For each image in which the object is tracked, it is determined whether the object appears “well-defined”. In particular, application 115 determines if the object is intact and whole in the respective image, i.e., the object appears complete and fully visible without missing or obstructed portions, or if the object is not intact, i.e., at least part of the object is missing or obstructed or not clearly visible. For example, a plant leaf (or other object) may appear (partially or fully) obstructed or concealed in a particular image frame due to the presence of one or more other objects, such as another plant leaf of the same or different plant, such as due to the position and orientation at which the image was captured and the conditions during imaging. For another example, a plant leaf may appear unclear (i.e., blurry or distorted) or otherwise not fully perceptible, such as due to fogginess or heavy rain or snow or other environmental or climate conditions present at the time of imaging.

If the object is considered not to be well-defined or characterized with a visually undetectable portion in a given image, then application 115 may supplement the missing, obstructed or concealed portions of the object. Supplementing visually undetectable object portions may be performed using various tools or techniques, such as by extracting the missing/obstructed/concealed portion from other image frames in which the same or similar object appears intact, with suitable modifications to account for differences between the image frames. An alternative procedure is to generate a bounding box around the object, which may not supplement or complete the whole object but may be used to evaluate the object dimensions. It is not always necessary to supplement all missing or obstructed image portions of an object in a particular image, only enough to enable subsequent phenotype determination. In certain cases it may be deemed unnecessary to supplement undefined object portions altogether. For example, each object may be assigned (e.g., using a pre-trained neural network model) a weighting or “intactness metric” reflecting the degree to which the object is well-defined in a particular image, such as a coefficient between 0 and 1, with “0” representing “completely not intact” and “1” representing “completely intact”, and if the weighting is above a selected threshold (e.g., above 0.8) then the object is deemed sufficiently well-defined and supplementation not required. This criterion may further be associated with a reliability metric for object detection, as will be further discussed hereinbelow. If the object is determined to be well-defined in a given image, then phenotyping may be performed based on the original object as it appears. The degree to which an object is well-defined in an image may also be utilized in determined supplemental point cloud distances, such that a supplemental distance is performed for images containing the object in a maximal well-defined state (i.e., having an intactness metric above a certain threshold).

Application 115 processes the images in which a tracked object appears and determines one or more phenotypes of the object. The phenotypes may include various traits or properties of the object, including but not limited to: size, shape; dimensions (e.g., height, width, length), volume, density, color, regularity, uniformity, developmental stage, amount of plant organs (e.g., fruit) per unit of area or field of view section, presence/absence of pests or diseases or plant disorders, and the like. Certain traits may be relevant only for certain object levels in the plant hierarchy, for example certain phenotypes may be applicable for plant organs (e.g., size, volume or regularity of a fruit or leaf), and other phenotypes may be applicable only for plants or plant groupings or an entire field (e.g., number or density of fruits in an individual plant or in a plant segment or field area). The phenotype determination may utilize the distance values (point cloud coordinates and/or supplemented distance coordinates) associated with the object, and the degree to which the object is well-defined in a given image. The object tracking process may be used to facilitate a phenotype determination. For example, a Kalman filter estimation process may be used to determine whether a single fruit cluster is split in a given image frame, and to correctly count the number of clusters in a field of view (FOV) and avoid confusion between a cluster brunch at a given position and a number of clusters in the same position. The phenotype determination may also account for environmental information associated with the agricultural scene at the time of image capture. For example, application 115 may receive and take into account factors such as: temperature (e.g., obtained using a thermometer or temperature sensor); humidity (e.g., obtained using a humidity sensor); ambient lighting (e.g., obtained using a light sensing sensor); presence of atmospheric contaminants, and the like.

Each determined phenotype is associated with a “reliability metric”, reflecting a degree of confidence or reliability of the phenotype accuracy. For example, an object may be assigned a first phenotype (e.g., fruit size value) having a relatively high reliability metric (e.g., >90%), and assigned a second phenotype (e.g., uniformity value) having a relatively low reliability metric (e.g., <80%). The reliability metrics may be further fitted according to various criteria, such as the number and reliability of distance coordinates obtained for that object over the tracked images. Other criteria may include: number or degree of obstructions of the object (e.g., plant organ) over the tracked images; the size, distance or regularity of the object (e.g., plant organ) over the tracked images; imaging characteristics, such as image resolution, sensitivity, field of view, lens type, spectral range; and the like.

The aforementioned process (object detection and tracking, generating supplemental distance data if needed, generating supplemental image portions if needed, phenotype determination associated with respective reliability metrics) may be iteratively repeated for multiple objects within the captured images, first within a given object level and then for higher object levels. For example, the process is repeated for multiple plant organs, such as for different fruits (and/or leaves, stems, roots) within an individual plant crop. Subsequently, the process is repeated for multiple plant organs within a different plant crop, and so forth. After sufficient phenotypes are obtained for different plants, the same process can be repeated for multiple plants within an individual plant grouping, and so forth. Finally, when sufficient phenotypes are obtained for different plant groups, the same process can be repeated for multiple plant groups within a segment of the field and eventually for the entire field. It is noted that a captured image may include multiple objects of interest, such as multiple plant organs (e.g., fruits) of an imaged plant, and accordingly the processing of different objects in one or more images may be performed simultaneously or successively.

Multiple objects may be grouped or classified into categories based on common features or attributes, and phenotype statistics may be determined for one or more objects and/or object categories. For example, a collection of plants in a certain plant group may be assigned to a first category (e.g., an apple crop of a first apple variant), whereas a different collection of plants in the same plant group may be assigned to a second category (e.g., a second apple variant). Phenotypes may then be determined for each category based on the individual phenotypes of the objects belonging to that category. Subsequently, phenotype statistical metrics and distributions may be determined, for one or more objects or object categories, taking into account the reliability metrics of the various phenotypes within the category. Phenotype information may also be determined for selected temporal durations, such as depicting changes in phenotype distribution over days, weeks, months, years, calendrical seasons (e.g., autumn, spring) or agricultural seasons (e.g., planting period, harvesting period). For example, determined group level statistics may include uniformity and development of one or more plant groupings over a selected duration.

The phenotype statistics and distributions may be indicated to the user, such as via application 115 providing a plant phenotyping report on user computing device 110, which may include a visual representation of values or graphs presented on display 116. The phenotyping report may include an indication of the reliability metrics associated with respective phenotypes for respective objects and object categories. Application 115 may also provide additional information, such as historical phenotype statistics obtained for the same field on previous dates and times, and/or corresponding data obtained for similar fields or plant types in other fields by other users. Phenotype statistics may also be presented over a selected duration, such as depicting changes in phenotypes over days, weeks, months, years, seasons, agricultural seasons, and the like. Based on the presented phenotype report, the user can decide how to improve or optimize crop development of particularly plants or plant groups within a particular field. Application 115 may also provide recommendations for enhancing crop development in accordance with the obtained information. The user may specify particular attributes of crop development to optimize (e.g., to maximize the number or size of fruits within a selected plant grouping; or to minimize the number of diseased fruits within a selected plant grouping), and application 115 may determine and present recommendations geared for optimizing the requested criteria. Application 115 may further guide the user when imaging a targeted object, such as to provide a recommended imaging trajectory, in accordance with a spatial distance of the targeted object, such as based on how close or far camera 112 is positioned and directed relative to the target object, for optimizing the captured images.

Plant phenotype application 115 may utilize machine learning techniques to determine relevant phenotype traits and to identify patterns for classifying objects and phenotypes into categories. The data analysis may utilize any suitable machine learning approach or algorithm known in the art, including but not limited to: an artificial neural network (ANN) algorithm, such as a recurrent neural network (RNN); a deep learning algorithm; a linear regression, logistic regression or other regression model; and/or a combination thereof. The data analysis may utilize any suitable tool or platform, such as publicly available open-source machine learning or deep learning tools.

It will be appreciated that the disclosed embodiments may allow for plant phenotyping with a high degree of sensitivity, irrespective of plant population, field type, or field conditions. For example, the present disclosure may provide a degree of sensitivity capable of providing phenotype measurements of plant organs having a size greater than 2 mm, and capable of identifying at least 10% of changes in the amount of plant organs within a plant population or plant group in a particular field. Furthermore, the plant phenotyping method and system of the present disclosure may achieve a high degree of accuracy (e.g., at least 80% for field related phenotyping and at least 90% for plant and plant organ phenotyping) and a low error rate (e.g., below 20%), irrespective of plant population, field type or field conditions. Furthermore, the plant phenotyping method and system of the present disclosure may be achieved using readily available devices, such as a basic smartphone with standard built-in components, requiring no more than a camera, inertial measurement sensors, and processor. The phenotyping method and system of the present disclosure may allow for differentiating between crop fruits (or other plant organs) in high density clusters, and between those that appear highly similar but contain exceedingly subtle distinctions, while accounting for scenarios such as merged or split plant organs, or overlapping portions of multiple plants, that may otherwise lead to erroneous or inaccuracies in phenotyping. The obtained phenotype parameters can be assigned to different plant classifications and categories along multiple hierarchical levels, ranging from an individual plant organ, to an individual plant or crop, to multiple plants in a plant grouping, to multiple plant groups in a field, for obtaining pertinent statistical information of the determine phenotypes that can be utilized for enhancing crop development at varying stages of growth. The phenotyping may be adapted to particular requirements of the user, such as based on the type of crop or type of field or other criteria, allowing the user to indicate which phenotypes or (phenotype category statistics) are of particular interest and/or which plant properties should be taken into account when determining a general plant phenotype (e.g., which plant organs), and can assign different values or weightings to certain lower level phenotypes (e.g., for plant organs or plants) when establishing higher level phenotypes (e.g., for plant groupings or a field). The phenotypes and phenotype statistics may be determined and fine-tuned based on machine learning of large collections of data (e.g., using a remote cloud-based computing platform), such as historical phenotype data associated with the same or similar type of crop or field, including data obtained from the same user or other users. The disclosed plant phenotyping system and method may further provide recommendations for improving crop development based on the determined phenotype parameters and statistics. These recommendations may also be dynamically updated and optimized via machine learning of relevant historical data collections, such as previous recommendations accounting for previous phenotyping of the same or similar crop or field types, such as using a remote cloud-based computing platform.

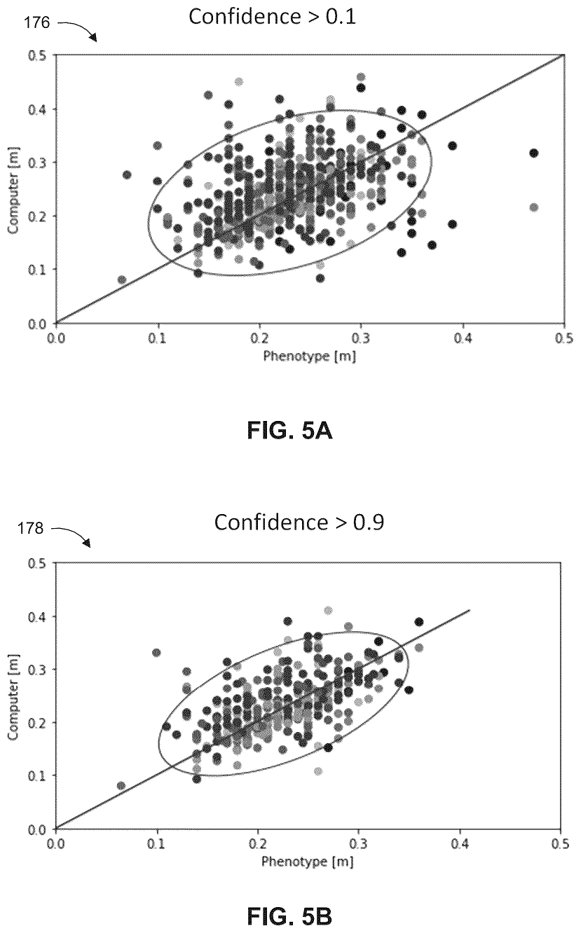

The disclosed plant phenotyping method and system may provide accurate image-based phenotyping measurements which are comparable to manually obtained physical measurements. Reference is made to FIGS. 5A and 5B, which respectively show a first graph 176 (FIG. 5A) and a second graph 178 (FIG. 5B), showing an exemplary phenotype measurement for grape cluster length obtained by a method in accordance with a disclosed embodiment, as a function of the same phenotype obtained manually, for different reliability metrics. In graph 176, the y-axis represents grape cluster size (length) phenotype measurements obtained by the disclosed image-based plant phenotyping method for a reliability metric above 0.1, and the x-axis represents corresponding grape cluster length phenotype measurements obtained manually. In graph 178, the y-axis represents similar grape cluster size (length) phenotype measurements obtained by the disclosed image-based plant phenotyping method plot for a reliability metric above 0.9. The reliability metric for this phenotype refers to the ability to detect a grape cluster as a whole (intact). It is apparent that the plotted values of second graph 178 are clustered together closer along the diagonal line representing a perfect (100%) correlation between the manual and the image-based phenotype measurements, as compared to the plotted values of first graph 176, indicating that the correlation is higher for the higher reliability metric.

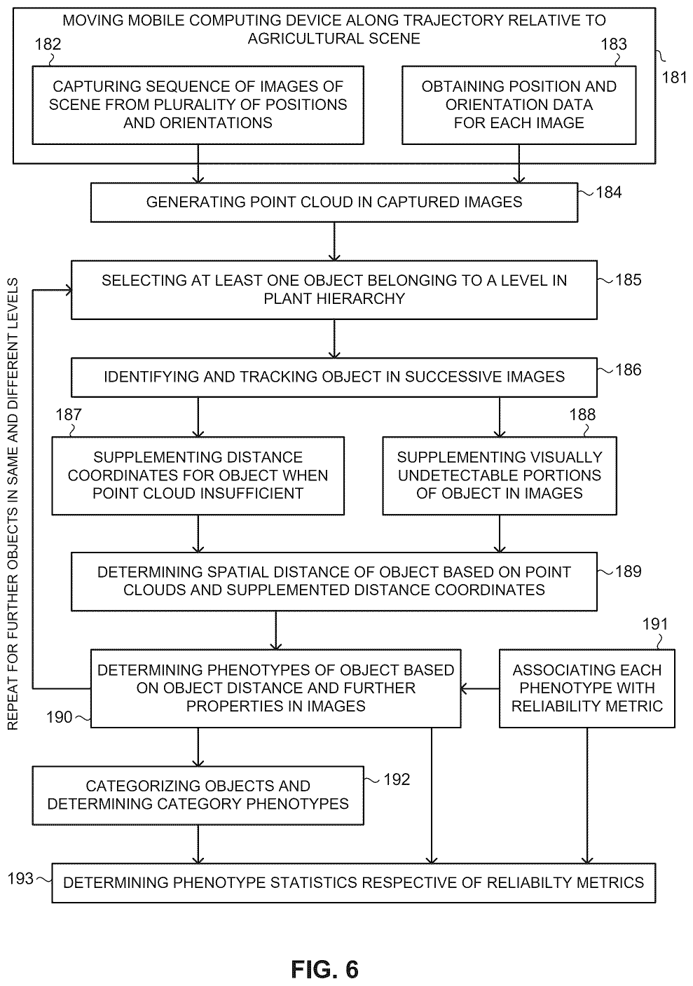

Reference is now made to FIG. 6, which is a flow diagram of a method for plant phenotyping over a computer network, operative in accordance with an embodiment of the present disclosure. In procedure 181, a mobile computing device is moved along a trajectory relative to an agricultural scene, and in procedures 182 and 183, a sequence of images of the agricultural scene is captured from a plurality of relative positions or orientations along the trajectory, using at least one image sensor of the mobile computing device, and position and orientation data along the trajectory is obtained using an inertial measurement unit of the mobile computing device. Referring to FIGS. 1 and 2, user computing device 110 is moved along a path 140 relative to a plant field 200 containing multiple plants 205 arranged in plant grouping 210. For example, referring to FIG. 3, user computing device 110 is conveyed along a trajectory 150, and camera 112 captures a sequence of images (161, 162, 163) of a crop 205 at a plurality of trajectory points (150A, 150C, 150E) along trajectory 150 at respective positions and orientations relative to crop 205. Camera 112 is positioned at a minimum imaging distance, which may be a function of a plant size or plant organ size, such that, for example, the plant or plant organ occupies a minimum number of image pixels in the captured images.

In procedure 184, a point cloud is generated for each captured image. Referring to FIGS. 1 and 2, phenotyping application 115 processes the captured images and associated image metadata and position and orientation data to establish a point cloud in the images. The point cloud includes at least one 3D spatial coordinate defining at least a distance coordinate respective of features in the imaged scene. The point cloud may be generated by a process operating simultaneously to the image and inertial data capturing, or by serial processing of image frames. Generation of point cloud coordinates may utilize known techniques, such as visual inertial odometry (VIO). The amount and location of point cloud coordinates may vary among images in a captured sequence, as well as within an individual image, such that certain image portions or certain images may be associated with relatively few point cloud coordinates.

In procedure 185, at least one object is selected, where the object belongs to a level in a plant hierarchy. Referring to FIGS. 1 and 2, phenotyping application 115 selects a particular feature or object of interest (OOI) in agricultural scene 200 for analysis. The object belongs to an object level in a plant hierarchy, which includes (in increasing hierarchical level order): a plant organ, an individual plant; a plant group; and a plant field. For example, in a first iteration, phenotyping application 115 selects a plant organ, such as a leaf or fruit of plant 205, whereas in a subsequent iteration, application 115 selects an entire plant 205.

In procedure 186, the selected object is identified and tracked in successive images of the captured image sequence. Referring to FIGS. 1-3, phenotyping application 115 processes the captured images and associated position and orientation data and image metadata, to identify and track the selected object, such as a plant organ (e.g., a leaf or fruit of plant 205) in images 231, 232, 233 captured from different positions and viewing angles.

In procedure 187, distance coordinates for the object are supplemented in the images when the point cloud coordinates is insufficient. referring to FIG. 1, phenotyping application 115 processes the captured image sequence and determines for each image whether insufficient point cloud coordinates, particularly distance coordinates, fall on the image regions containing the OOI to allow for determination of the OOI distance in the respective image. the existing point cloud may be insufficient from either a quantity standpoint (e.g., too few coordinates) or from a quality standpoint (e.g., coordinates unusable or of low quality). if there are insufficient point cloud coordinates, phenotyping application 115 may supplement distance of the OOI, using at least one suitable technique, such as: LIDAR range measurements, stereoscopic rangefinder measurements, extrapolation based on average or accepted distances for same or similar objects or based on distance to same or similar objects in other images. A distance measurement may be updated in accordance with the location of an imaging trajectory of camera 112 when capturing the images, where the imaging trajectory may be reconstructed based on the captured images and/or associated inertial data.

In procedure 188, visually undetectable portions of the object are supplemented in the images. Referring to FIGS. 1-3, phenotyping application 115 processes the images (231, 232, 233) and determines if the tracked OOI is intact in a respective image, or if the OOI includes portions that are missing, obstructed, concealed, or otherwise not visually detectable. For example, a plant organ may be partially or fully concealed in a respective image by other organs or plant features in the image which obstruct its view, such as due to the position and orientation at which the image was captured and/or environmental conditions during imaging. If such visually undetectable object portions are identified, phenotyping application 115 may supplement missing, obstructed or concealed portions of the OOI, as necessary. For example, phenotyping application 115 may extract a missing or obstructed portion from other image frames in which the same or similar object is intact, with suitable modifications, such as to reconstruct a more complete representation of the object from multiple images. In a further example, a pre-trained neural network algorithm may be applied to create a bounding box around the OOI and evaluating the bounding box dimensions. The OOI may be assigned an intactness metric reflecting the degree to which the object is determined to be well-defined in a given image, such as a coefficient between 0 and 1, where “0” represents completely obstructed or concealed (i.e., 100% not well-defined or completely visually undetectable) and “1” represents completely intact (i.e., 100% well-defined or completely visually detectable). If the reliability metric is above a selected threshold coefficient value (e.g., above 0.8), then the object is deemed sufficiently well-defined and supplementation of visually undetectable object portions is not required. If the OOI is determined to be well-defined in a given image, then phenotyping may be performed based on the object representation in the image.

In procedure 189, a spatial distance to the object is determined based on point clouds in the image and the supplemented distance coordinates. Referring to FIG. 1, phenotyping application 115 processes the images (231, 232, 233) and determines a spatial distance of the OOI (in 3D space). The object distance is determined based on point cloud coordinates (generated in procedure 184) that fall on the OOI in the image, and based on supplemented distance coordinates (generated in procedure 185) when applicable. The object distance may be determined over successive images in the image sequence, without necessitating to generate a 3D model of the object.

In procedure 190, at least one phenotype of the object is determined. Referring to FIG. 1, phenotyping application 115 determines one or more phenotypes of the OOI, which may include traits or properties of the OOI (e.g., plant organ), such as size, dimensions, volume, density, color, regularity, uniformity, developmental stage, amount of organs, presence of pests or diseases, and the like. The plant phenotypes may be determined based on at least the determined distance of the OOI in the tracked images, as well as other properties of the OOI, and the degree to which the object is well-defined in a given image. An object tracking process may be used to facilitate a phenotype determination. Certain phenotypes may be dependent on the plant hierarchy object level, such as plant organ phenotypes (e.g., size or volume or regularity of a plant fruit, and plant cluster phenotypes (e.g., number or density of fruits in a cluster). The determined phenotype may account for environmental information, such as temperature, humidity, ambient lighting, which may be obtained from environmental sensors.

In procedure 191, each determined phenotype is associated with a reliability metric. Referring to FIG. 1, phenotyping application 115 establishes a reliability metric for each phenotype, reflecting a degree of confidence or reliability of the phenotype accuracy for a given OOI. For example, an OOI may be assigned a first phenotype (e.g., fruit size) with a high reliability metric (e.g., above 90%) and a second phenotype (uniformity of fruits) with a low reliability metric (e.g., below 80%). The reliability metrics may be fitted according to suitable criteria, such as the number and reliability of distance coordinates obtained for that OOI over the tracked images; number and degree of obstructions over the tracked images; imaging characteristics relating to the captured images; and the like. An indication of determined phenotypes may be provided to the user, such as via display 116 of user computing device 110. At least some of the aforementioned method steps (procedures 184, 185, 186, 187, 188, 189, 190, 191) may be iteratively repeated for multiple objects, at the same and other object levels in the plant hierarchy. For example, the method steps may be iteratively repeated for multiple plant organs, such as for different fruits of an individual plant, then iteratively repeated for multiple plant organs of different plants, then for multiple plant groups, and finally for an entire plant field.

In procedure 192, objects are grouped into categories and at least one category level phenotype is determined. Referring to FIG. 1, phenotyping application 115 classifies a plurality of objects into one or more categories based on common features or attributes, and determines phenotypes or phenotype statistics for a respective category. For example, phenotyping application 115 may group different plants according to their fruit variants, determine a first set of phenotypes for a first plant group belonging to the first plant variant, and determine a second set of phenotypes for a second plant group belonging to a second plant variant.

In procedure 193, at least one phenotype statistical metric is determined for at least one object or object category in accordance with a respective reliability metric of at least one phenotype of an object in the category. Referring to FIG. 1, phenotyping application 115 determines phenotype statistical metrics and distributions for one or more objects in accordance with the reliability metrics reflecting respective confidence levels of one or more phenotypes of the objects. Phenotyping application 115 may further determine statistical metrics and distributions for one or more object categories in accordance with the reliability metrics of the phenotypes of objects belonging to that category. Phenotype statistics may be determined for selected temporal durations, such as depicting changes in phenotype distribution over days, weeks, months, years, calendrical seasons, or agricultural seasons. Phenotyping application 115 may provide a report with analysis, insights and recommendation for improving or optimizing plant growth in accordance with the determined phenotype statistics. A report may further include an indication of reliability metrics of respective phenotypes for respective objects and categories; historical phenotype statistics relating to the same or similar plant field, plant grouping, plant, or plant organ; and phenotype statistics filtered over a selected temporal duration. The user may provide criteria for enhancing crop growth and development, such as in order to optimize selected crop phenotypes (e.g., to maximize the number or size of fruits within a selected cluster, or to minimize the number of diseased fruits within a selected cluster), and phenotyping application 115 may determine and provide recommendations intended for optimizing the user criteria.

While certain embodiments of the disclosed subject matter have been described, so as to enable one of skill in the art to practice the present disclosure, the preceding description is intended to be exemplary only. It should not be used to limit the scope of the disclosed subject matter, which should be determined by reference to the following claims.

1. A method for plant phenotyping, comprising the procedures of:
capturing a sequence of images of an agricultural scene from a plurality of positions and orientations along a trajectory using at least one image sensor of a mobile computing device, and obtaining position and orientation data for each captured image using at least one inertial sensor of the mobile computing device;
generating a point cloud in each of the captured images, the point cloud comprising at least one coordinate defining a distance from the image sensor to a respective feature in the image;
selecting at least one object belonging to a plant hierarchy level comprising one of: a plant organ; a plant; a grouping of plants; and a field of plants; and
for each of at least one first selected object in a first plant hierarchy level, applying the processing steps of:
identifying and tracking the object in successive images of the sequence;
supplementing distance coordinates for the object when the point cloud is insufficient;
supplementing visually undetectable portions of the object in the images;
determining a spatial distance of the object in respective images based on the point cloud coordinates and the supplemented distance coordinates; and
determining at least one phenotype of the object, based on the determined spatial distance of the object.

2. The method of claim 1, further comprising the procedures of determining at least one phenotype statistic based on a reliability metric reflecting a degree of confidence or reliability of the determined phenotype, the reliability metric based on at least one parameter selected from the group consisting of number or reliability of distance coordinates for object; number of plant organ obstruction; number or degree of obstructions of object in images; size, distance or regularity of object in images; and imaging characteristics on the image sensor.

3. The method of claim 1, further comprising the procedure of determining at least one phenotype or phenotype statistic based on a grouping of a plurality of objects into at least one category.

4. The method of claim 1, comprising selecting at least one second selected object in a second plant hierarchy level, and applying the processing steps for the second selected object.

5. The method of claim 1, further comprising the procedure of providing a phenotyping report on a phenotype application executing on the mobile computing device, the phenotyping report comprising information relating to at least one determined phenotype.

6. The method of claim 1, further comprising the procedure of obtaining environmental data of the agricultural scene, wherein at least one of the procedures of: supplementing distance coordinates; supplementing visually undetectable portions of the object; and determining at least one phenotype, is performed based on the environmental data.

7. The method of claim 1, wherein the procedure of supplementing distance coordinates is performed using at least one technique selected from the group consisting of:
Light Detection and Ranging (LIDAR);
stereoscopic imaging;
determining an average or accepted distance for a same or similar object; and
extrapolating from distance information in at least one other image.

8. The method of claim 1, further comprising the procedure of reconstructing an imaging trajectory of the image sensor when capturing the sequence of images, using the position and orientation data or the captured images, and using the reconstructed imaging trajectory for at least one of:
determining or updating a distance measurement from the image sensor to the object;
correcting a position and orientation of the image sensor; and
determining an alignment of an arrangement of plants.

9. The method of claim 1, further comprising the procedure of providing at least one recommendation for optimizing crop development in accordance with the determined phenotype information.

10. The method of claim 1, wherein the phenotype comprises at least one attribute selected from the group consisting of: size; shape; dimensions; volume; amount; density; color; regularity; uniformity; developmental stage; and presence or absence of at least one pest or at least one disease or plant disorder.

11. (canceled)

12. The method of claim 1, wherein at least one of the processing steps of: supplementing distance coordinates for the object; and supplementing visually undetectable portions of the object, is applied based on an intactness metric reflecting a degree to which the object is well-defined in a respective image.

13. (canceled)

14. (canceled)

15. The method of claim 1, further comprising the procedure of guiding the imaging of an object in accordance with the spatial distance of the object in relation to the imaging sensor of the mobile computing device.

16. A system for plant phenotyping, the system comprising:
a mobile computing device, communicatively coupled to a computer network, the mobile computing device comprising:
at least one image sensor, configured to capture a sequence of images of an agricultural scene from a plurality of positions or orientations along a trajectory relative to the agricultural scene; and
at least one inertial sensor, configured to obtain position and orientation data along the trajectory;
the system further comprising a processor configured to generate a point cloud in each of the captured images, the point cloud comprising at least one coordinate defining a distance from the image sensor to a respective feature in the image, and to select at least one object belonging to a plant hierarchy level comprising one of: a plant organ; a plant; a grouping of plants; and a field of plants, and
for each selected first object in a first plant hierarchy level, the processor is further configured to: identify and track the object in successive images of the sequence, to supplement distance coordinates for the object when the point cloud is insufficient, to supplement visually undetectable portions of the object in the images; to determine a spatial distance of the object in respective images based on the point cloud coordinates and the supplemented distance coordinates, and to determine at least one phenotype of the object, based on the determined spatial distance of the object.

17. The system of claim 16, wherein the processor is configured to determine at least one phenotype statistic based on a reliability metric reflecting a degree confidence or reliability of the determined phenotype, the reliability metric based on at least one parameter selected from the group consisting of: number or reliability of distance coordinates for object; number of plant organ obstructions: number or degree of obstructions of object in images: size distance or regularity of object images: and imaging characteristics of the image sensor.

18. The system of claim 16, wherein the processor is configured to determine at least one phenotype or phenotype statistic based on a grouping of a plurality of objects into at least one category.

19. The system of claim 16, comprising selecting at least one second selected object in a second plant hierarchy level, and applying the processing steps for the second selected object.

20. The system of claim 16, further comprising a phenotype application executing on the mobile computing device, the phenotype application configured to provide at least one of:
a phenotyping report comprising information relating to at least one determined phenotype;
at least one recommendation for optimizing crop development in accordance with the determined phenotype information; and
at least one recommendation for guiding the imaging of an object in accordance with the spatial distance of the object in relation to the imaging sensor of the mobile computing device.

21. The system of claim 16, further comprising at least one environmental sensor, configured to obtain environmental data of the agricultural scene, wherein at least one of: supplementing distance coordinates; supplementing visually undetectable portions of the object; and determining at least one phenotype, is performed based on the environmental data.

22. (canceled)

23. The system of claim 16, wherein the processor is further configured to reconstruct an imaging trajectory of the image sensor when capturing the sequence of images, using the position and orientation data or the captured images, and use the reconstructed imaging trajectory for at least one of:
determining or updating a distance measurement from the image sensor to the object;
correcting a position and orientation of the image sensor; and
determining an alignment of an arrangement of plants.

24-29. (canceled)
